(19) United States
(12) Patent Application Publication
(10) Pub. No.: US 2005/0070461 A1
(43) Pub. Date: Mar. 31, 2005

(54) Means and methods for altering the motility of the gastrointestinal tract

(76) Inventors: Guy Eduard Elisabeth Boeckxstaens, Bussum (NL); Wouter Jacob de Jonge, Ouderkerk Aan De Amstel (NL)

Correspondence Address: TRASK BRITT, P.O. BOX 2550, SALT LAKE CITY, UT, 84110, US

(21) Appl. No.: 10/921,490

(22) Filed: Aug. 18, 2004

(30) Foreign Application Priority Data
Feb. 18, 2003 (WO) PCT/NL03/00120
Aug. 21, 2003 (WO) 03/068261
Feb. 18, 2002 (EP) 02075643.3

(51) Int. Cl. A61K 38/00

(52) U.S. Cl. 514/2

(57) ABSTRACT

Provided is a use of a compound capable of influencing, at least in part, an activity of a neural reflex pathway for the preparation of a medicament for altering the motility of the gastrointestinal tract. The medicament can be used for prophylaxis and/or treatment of hypomotility of the gastrointestinal tract. Preferably, generalized hypomotility occurring during postoperative ileus is prevented and/or treated by a medicament of the invention. In one aspect, the neural reflex pathway's activity is decreased by, at least in part, preventing stimulation of the pathway by an immunocyte, a macrophage and/or a mast cell. Activity can also be decreased by, at least in part, preventing immunocyte recruitment. A compound capable of, at least in part, influencing an activity of, for instance, a neural reflex pathway comprises an anti-ICAM-1 antibody, an anti LFA-1 antibody, and/or ketotifen. A pharmaceutical composition for prophylactic and/or therapeutic treatment of an individual against hypomotility of the gastrointestinal tract comprising a compound capable of, at least in part, decreasing a neural reflex pathway is also herewith provided.

### CROSS-REFERENCE TO RELATED APPLICATION

This application claims priority to International Application No. PCT/NL03/00120, filed Feb. 18, 2003, published in English as PCT International Publication No. WO 03/068261 on Aug. 21, 2003, the contents of which are incorporated by this reference.

### TECHNICAL FIELD

The invention relates generally to the field of medicine. More specifically, the invention relates to the field of treatment of gastrointestinal disorders.

### BACKGROUND

Abnormalities in gastrointestinal motility and visceral sensation form the basis of a wide range of gastrointestinal diseases, such as gastroesophageal reflux disease, functional dyspepsia, irritable bowel syndrome (IBS) and postoperative ileus. The invention especially relates to the latter. Postoperative ileus is characterized by postoperative dysmotility of the gastrointestinal tract that occurs after essentially every abdominal operation. This effect, which can last for days, contributes to postoperative discomfort and increases morbidity due to impaired gastrointestinal motility. The delayed resumption of oral intake is one of the major causes of a prolonged hospital stay after abdominal surgery.

Current research of gastrointestinal motility abnormalities has revealed that postoperative ileus involves two phases of dysmotility: an initial phase and a second, sustained phase. Most previous experimental animal research was focused on the pathophysiology of instant hypomotility during or directly after abdominal surgery (1-4). It is currently widely accepted that the acute hypomotility of the gastrointestinal tract during small bowel manipulation results from the activation of inhibitory neural pathways, both adrenergic and non-adrenergic in nature. Activation of mechanoreceptors and nociceptors during surgery triggers sensory splanchnic nerve fibers, synapsing in the prevertebral ganglia or in the spinal cord, to activate inhibitory adrenergic nerves (1). In addition to these spinal reflexes, intense painful stimuli can activate medullary, pontine, and hypothalamic nuclei (5), and trigger supraspinal inhibitory reflexes, resulting in acute generalized postoperative ileus.

The initial process of activation of inhibitory neuronal reflex pathways wanes early after surgery. However, resumption of peristalsis in humans generally takes 3 to 5 days (6), indicating that the initial phase is followed by a second, sustained phase of dysmotility. Obviously, preventing the occurrence of this late phase is clinically the most relevant. The mechanism underlying this sustained component of postoperative ileus, which develops after cessation of mechanical or nociceptive stimuli, cannot be mediated through stimulation of mechano-receptors, as it is known that afferent nerves, activated by mechanoreceptors, only fire during the mechanical activation (7). Recently, it was reported that surgical manipulation of the small bowel elicits the establishment of a leukocyte infiltrate, mainly consisting of neutrophils, in the intestinal muscularis externa (4, 8). The presence of the infiltrate was associated with an impaired in vitro contractility of muscle strips of the affected intestinal tissue. Inhibition of leukocyte recruitment via postoperative blocking of ICAM-1/LFA-1 interaction normalized this local contractility (9). The knowledge that postoperative administration of non-steroidal anti-inflammatory agents reduce the sustained ileus in humans (10) and rats (11) corroborate to the hypothesis that an inflammatory component may be involved in the pathogenesis of postoperative ileus. These findings are, however, only concerned about impaired local contractility of the manipulated intestinal tissue. They do not explain the generalized hypomotility seen in postoperative ileus, which involves the whole gastrointestinal tract. The local action of a leukocyte infiltrate cannot explain a motility disorder of a gastrointestinal part which is located at a site which is (anatomically) away from the leukocyte infiltrate.

Hence, in spite of interesting research results obtained in the field, the pathophysiological mechanisms responsible for abnormalities in gastrointestinal motility, especially abnormalities of sustained general hypomotility of the gastrointestinal tract, are still not fully known. Furthermore there is a lack of efficient treatment. Current treatment is restricted to conservative methods, such as nasogastric suction, or the use of prokinetic agents, which have significant side effects. The success of shortening the duration of postoperative ileus by these strategies is limited and, clearly, an effective treatment of gastrointestinal hypomotility would be highly desirable.

### SUMMARY OF THE INVENTION

Provided is the use of a compound capable of, at least in part, influencing an activity of a neural reflex pathway for the preparation of a medicament for altering the motility of the gastrointestinal tract. It is found in the present invention that decreased motility of the gastrointestinal tract during the sustained second phase of postoperative ileus has an inflammatory origin that results in the triggering of at least one inhibitory neural reflex pathway via a neuro-immune interaction. Therefore, a compound capable of influencing an activity of the neural reflex pathway is capable of influencing the motility of the gastrointestinal tract. Preferably, activity of the neural reflex pathway is decreased. In postoperative ileus, decrement of an activity of a neural reflex pathway results in a normalization of gastrointestinal motility.

By an “activity of a neural reflex pathway” is meant herein activation/depolarization of a sympathetic or parasympathetic neuron that synapses in prevertebral ganglia, spinal cord and/or brain stem, and that projects directly back onto a specific organ. This neuron may, for instance, be cholinergic, adrenergic or non-cholinergic/non-adrenergic in nature.

By “activation of the neural reflex pathway” is meant herein activation/depolarization of the neuron, preferably initiated by infiltration and/or activity of leukocytes at the peripheral synapses.

By the motility of the gastrointestinal tract is meant the cap ability of the gastrointestinal tract of processing nutriments and forcing nutriments (which may or may not be at least partly processed) to flow through the tract. This is mainly due to the activity of smooth muscle cells. A motility of the gastrointestinal tract can involve the whole gastrointestinal tract or a part of the tract, like, for instance, the stomach, (part of) the small intestine and/or (part of) the colon.

Since it is now possible according to the invention to use a compound capable of influencing a neural reflex pathway to influence the motility of any part of the gastrointestinal tract, the compound can be used for treatment of gastrointestinal disorders. Particularly, hypomotility of the gastrointestinal tract that results from local inflammation in the gastrointestinal tract can be very well treated with a compound of the invention. To achieve this goal, a medicament can be prepared comprising the compound and, for instance, a suitable carrier. The medicament can be administered to an animal or a human individual, for instance, before the gastrointestinal tract is manipulated. In that case, the medicament can serve as a prophylactic against sustained gastrointestinal hypomotility. The medicament can also be administered after a gastrointestinal operation is performed. The medicament can then still serve as a prophylactic, because the second stage of postoperative ileus starts several hours after surgery. Additionally, the medicament can be used to treat a patient that is already suffering from gastrointestinal hypomotility. Thus, in one aspect, the invention provides a use of a compound capable of, at least in part, altering activation of a neural reflex pathway for the preparation of a medicament for prophylaxis and/or treatment of hypomotility of the gastrointestinal tract. Prophylaxis and/or treatment can shorten the duration of postoperative ileus, as compared to untreated individuals. Prophylaxis with a compound of the invention can also, at least in part, prevent the occurrence of hypomotility. Methods to prepare a medicament are well known in the art and need no further explanation here.

Preferably, the hypomotility comprises a generalized hypomotility. By a “generalized hypomotility” is meant herein a hypomotility which occurs in at least part of the gastrointestinal tract that is not directly affected by human manipulation. Hypomotility often also occurs in a part of the gastrointestinal tract that is directly affected by human manipulation. Generalized hypomotility often involves hypomotility of the entire gastrointestinal tract, including parts which are either directly, or not directly affected by human manipulation. Human manipulation, for instance, comprises surgery or bowel obstruction due to surgery. As an example, after surgery of the stomach, generalized hypomotility of the gastrointestinal tract can occur in (part of) the small intestine and/or colon. Of course, the part which is affected by human manipulation can as well be subject to hypomotility, although this is not necessary.

In the art it has been demonstrated that manipulation of the gut elicits recruitment of leukocyte infiltrates in the muscularis externa, thus impairing local smooth muscle contraction. In the present invention, we have demonstrated that the leukocyte infiltrate is also capable of triggering a neural reflex pathway, resulting in postoperative ileus. Stimulation of the neural reflex pathway by immunocytes results in hypomotility of at least part of the gastrointestinal tract. Thus, immunocytes are also capable of inducing generalized hypomotility. This was not expected because other, distant parts of the gastrointestinal tract are not likely to be directly affected by immunocytes present at a certain site of manipulation. According to the invention, however, immunocytes do have a capability of influencing distant parts of the gastrointestinal tract, by stimulating a neural reflex pathway. Stimulation results in hypomotility in at least part of a non-manipulated part of the gastrointestinal tract. Therefore, one way of influencing the motility of at least part of the gastrointestinal tract can be performed by influencing the effect of an immunocyte upon an activity of a neural reflex pathway. Preferably, stimulation of the neural reflex pathway by an immunocyte is at least in part prevented. One embodiment of the invention, therefore, provides a use of the invention, wherein activity of the neural reflex pathway is decreased by, at least in part, preventing stimulation of the pathway by an immunocyte. Preferably, the immunocyte comprises a leukocyte. Stimulation of the neural reflex pathway by an immunocyte can, for instance, at least partly be prevented with general neural blockers (such as tetradotoxin), a nicotinic receptor blocker (such as hexamethonium), or blockers of the sympathetic neurons (such as guanethidine). In the examples, it is, for instance, shown that hexamethonium is capable of preventing the development of gastroparesis in mice.

After manipulation of a part of the gastrointestinal tract, immunocyte recruitment is observed. These immunocytes appear to be involved in stimulation of an activity of a neural reflex pathway, resulting in impaired motility of the gastrointestinal tract. If immunocyte recruitment is, at least in part, prevented, stimulation of an activity of the neural reflex pathway is, at least in part, indirectly prevented as well. Thus, instead of directly influencing the stimulation of an activity of a neural reflex pathway by an immunocyte, stimulation can also be influenced indirectly by avoiding the presence of immunocytes. One embodiment of the invention, therefore, provides a use of the invention, wherein activity of the neural reflex pathway is decreased by, at least in part, preventing immunocyte recruitment. Preferably, leukocyte recruitment is, at least partly, prevented.

By “immunocyte recruitment” is meant herein the influx of immunocytes, preferably leukocytes, at a specific site. Hence, the local amount of immunocytes is strongly enhanced at the site. Immunocyte recruitment can be induced by pro-inflammatory mediators, such as, for instance, histamine, trypsine, tryptase, chymase, 5-hydroxytryptamine, IL8 and/or TNFα or IL1β. One source of pro-inflammatory mediators are mast cells which are known to lie in close vicinity to nerve fibers. Once these mast cells are activated, a mixture of substances is released affecting cells in their vicinity, but most importantly, initiating the process of influx of inflammatory cells. Additionally, macrophages are known to release pro-inflammatory mediators in case of injury. These macrophages can be residential in the gastrointestinal tissue, and/or be recruited from the periphery. Hence, immunocyte recruitment can be counteracted by, at least in part, decreasing the release of a pro-inflammatory mediator by a macrophage and/or mast cell. In the examples is shown, for instance, that counteracting activation of mast cells prevents immunocyte recruitment and the development of generalized gastrointestinal hypomotility. Thus, by counteracting immunocyte recruitment, stimulation of an activity of a neural reflex pathway is indirectly counteracted as well. The invention, therefore, provides in one aspect, a the method according to the invention, wherein activity of the neural reflex pathway is decreased by, at least in part, decreasing the release of a pro-inflammatory mediator by a macrophage and/or mast cell. Preferably, a use of the invention is provided wherein the pro-inflammatory mediator comprises histamine, trypsine, tryptase, chymase, 5-hydroxytryptamine, IL8 and/or TNFα or IL1β. Most preferably, a release of a pro-inflammatory mediator is decreased by ketotifen.

Besides releasing pro-inflammatory mediators, mast cells and macrophages are capable of interfering with an activity of a neural reflex pathway. Typically, activity is stimulated by macrophages and mast cells, for instance, by releasing mediators such as histamine, tryptase and/or tachykinins which are capable of directly sensitizing afferent nerve endings. Therefore, an activity of a neural reflex pathway can also be decreased by preventing, at least partially, stimulation of the pathway by a macrophage and/or mast cell. A use of the invention wherein activity of the neural reflex pathway is decreased by at least in part preventing stimulation of the pathway by a macrophage and/or mast cell is, therefore, also herewith provided. Preferably, stimulation of activity of a neural reflex pathway is prevented by, at least in part, preventing release of IL1β histamine, tryptase, and/or tachykinins by the macrophage and/or mast cell.

Many proteins are involved in immunocyte recruitment and/or activation of a neural reflex pathway by an immunocyte, macrophage and/or mast cell. Such a protein can, for instance, increase immunocyte recruitment and/or activation of the pathway. Alternatively, such a protein can inhibit immunocyte recruitment and/or activation of the pathway. Proteins known to be involved in immunocyte recruitment and/or activation of a neural reflex pathway by an immunocyte, macrophage and/or mast cell comprise:
integrins/selectins, which are important for leukocyte adherence, rolling, and diapedesis in tissue (such as ICAM-1 and -2, JAM-1, LFA-1 and -2, P-selectin glycoprotein-1, L-selectin, P-selectin, α4-β7 integrin, MadCAM, CD44, and CD 99);
neuropeptides (such as substance P, NK-1 receptor, CGRP, CGRP-receptor, VIP, VIP-receptor, neutral endopeptidase, neurotensin, neurotensin receptor, nerve growth factor and neurotropin-3);
mast cells, mast cell proteases and mast cell mediators (such as histamine, trypsine, tryptase, chymase, 5-hydroxytryptamine, tumor necrosis factor-α, stem cell factor, and proteases activating PAR-2);
complement-involving proteins (such as antithrombin III, thrombin, Tissue Factor, Factor X, factor Xa, proteases activating PAR-1, PAR-3, PAR-4, and C5a, PAF and PF-4),
chemokines (such as C10, Eotaxin, HCC-1, I-309/TCA-3, JE, MCP-1, 2, 3, MIP-1α, -β, RANTES, MIP-2α and β, CINC-1, CINC-2, IFNγ-inducible protein-10 (IP-10), Monokine induced by IFN (MIG), Epithelial Neutrophil-Activating Peptide-78 (ENA-78), Granulocyte Chemotactic Protein-2 (IL8), Growth-related oncogene-α, β, γ (GRO/MGSA), IL8, and Stromal Cell-derived Factor);
cytokines (such as TNF-α, granulocyte-stimulating factor, granulocyte/macrophage-stimulating factor, platelet-derived growth factor, macrophage-migration inhibitory factor, transforming growth factor-β, IL1, IL2, IL3, IL6, IL9, IL13, IL-10), or their receptors, such as 1) the hematopoetic receptor superfamily (cytokine receptor superfamily), especially the IL2Rα, β-chains, IL4R, IL3Rα, and β-chains, IL5Rα, β-chains, IL6R, gp130, IL9R, IL12R, G-CSFR, GM-CSFR, CNTFR, LIFR, EpoR, PRLR en GHR, 2) the interferon receptor superfamily (IFNR-SF), among which IFNα, β-R, IFNγR, IL10R, and Tissue factor, and 3) nerve growth factor receptor superfamily (NGFR-SF), among which the cytokine receptors NGFR, TNFR-1 (p55), TNFR-2 (p75), CD27, CD30, CD40, CD40L, and all mitogen-activated protein kinases p42, p44, and p38).

The capability of such a protein of influencing immunocyte recruitment and/or activating a neural reflex pathway can be altered by binding at least a functional part of the protein.

By a “functional part of the protein” is meant herein a part which is, at least in part, involved in the capability of the protein of influencing immunocyte recruitment and/or activating a neural reflex pathway.

Once the protein or functional part is bound, the influence of the protein or the part upon immunocyte recruitment and/or upon an activity of a neural reflex pathway is altered. For instance, an antibody or functional part, derivative and/or analogue thereof, capable of specifically binding ICAM-1 can alter the capability of ICAM-1 of binding to its ligand, LFA-1, on activated leukocytes. Hence, tissue recruitment of the leukocyte can be decreased by the anti-ICAM-1 antibody or functional part, derivative and/or analogue thereof. Likewise, an antibody or functional part, derivative and/or analogue thereof capable of specifically binding LFA-1 is capable of decreasing immunocyte recruitment. In a preferred embodiment, a combination of anti-ICAM-1 and anti-LFA-1 antibodies, or functional parts, derivatives and/or analogues thereof, is used.

The invention, therefore, provides a the method according to the invention, wherein the compound is capable of specifically binding at least a functional part of a protein which is involved in immunocyte recruitment and/or activation of a neural pathway by an immunocyte, macrophage and/or mast cell. In one embodiment, the compound comprises an antibody capable of specifically binding the at least functional part of a protein, or a functional part, derivative and/or analogue thereof. Preferably, the protein which is involved in immunocyte recruitment and/or activation of a neural pathway by an immunocyte, macrophage and/or mast cell comprises integrins ICAM-1, LFA-1, statin, PSGL-1, L-selectin, L-selectin receptor, P-selectin, P-selectin receptor, α4-β7 integrin, MadCAM, CD44, CD99, or other peptides such as Substance P, NK-1 receptor, CGRP, CGRP-receptor, VIP, VIP-receptor, neutral endopeptidase, neurotensin, neurotensin receptor, nerve growth factor and/or neurotrophin-3.

Preferably, the compound comprises an antibody capable of specifically binding ICAM, or a functional part, derivative and/or analogue thereof, and/or an antibody capable of specifically binding LFA-1, or a functional part, derivative and/or analogue thereof.

A functional part of an antibody is defined as a part which has the same kind of binding properties in kind, not necessarily in amount. By “binding properties” is meant the capability to specifically bind a target molecule. A functional derivative of an antibody is defined as an antibody which has been altered such that the binding properties of the molecule are essentially the same in kind, not necessarily in amount. A derivative can be provided in many ways, for instance through conservative amino acid substitution.

A person skilled in the art is well able to generate analogous compounds of an antibody. This can for instance be done through screening of a phage display library. Such an analogue has essentially the same binding properties of the antibody in kind, not necessarily in amount.

Immunocyte recruitment can also at least be partially decreased by a polysaccharide fucoidin, capable of specifically binding L-selectin and P-selectin. Also suitable for a use of the invention are anti-inflammatory drugs like glucocorticosteroids, annexine-1 peptides, nonsteroidal anti-inflammatory drugs (such as acetylsalicylic acid, ketorolac, ketoprofen, diclofenac, ibuprofen, and specific COX-2 inhibitors), antagonists of prostaglandins and bradykinins which affect gastrointestinal motility and cause leukocyte influx, inhibitors of 5-lipoxygenase activity, antagonists of LTB-4, and endogenous or exogenous opioids (such as endomorphin-1 and -2, endorphin, dynorphin, and hemorphin-7, or other μ, κ and δ agonists).

An activity of a neural reflex pathway can also be influenced by antagonizing the activation of mast cells. Hence, pharmaca that stabilize mast cells and/or antagonize actions of histamine released from mast cells and/or intervene with the activation or degranulation of mast cells are suitable for a use of the invention. Preferably, ketotifen, doxantrazole, or cromoglycates are used. More preferably, a use of the invention is provided wherein the compound comprises ketotifen. Immunocyte recruitment can also be prevented by an agent (such as, for instance, a nicotinic receptor agonist) or intervention (such as, for instance, vagal nerve stimulation by electrical stimulation or an agent such as CNI-1493) that inhibit macrophages and/or other inflammatory cells to release their substances.

Additionally, expression of a protein capable of influencing immunocyte recruitment and/or activating a neural reflex pathway can be altered. For instance, expression can be decreased. In that case, less protein will be present. If a protein capable of stimulating an activity of a neural reflex pathway is less expressed, the neural reflex pathway will as a result be less stimulated. Likewise, if a protein capable of increasing immunocyte recruitment is less expressed, the influx of immunocytes will be less. Expression of a protein can be altered with a nucleic acid capable of binding at least a functional part of a nucleic acid encoding the protein. Binding at least in part influences expression of the protein. The invention, therefore, provides a the method according to the invention, wherein the compound comprises a nucleic acid capable of binding at least a functional part of a nucleic acid encoding a protein which is involved in immunocyte recruitment and/or activation of a neural pathway by an immunocyte, macrophage and/or mast cell. Preferably, the compound comprises an antisense strand of at least a functional part of the nucleic acid, like, for instance, antisense ICAM-1.

In terms of the invention, a functional part of a nucleic acid is defined as a part of the nucleic acid, at least 30 base pairs long, preferably at least 200 base pairs long, comprising at least one expression characteristic (in kind, not necessarily in amount) as the nucleic acid. An antisense strand of a nucleic acid is defined as a nucleic acid molecule comprising a sequence which is essentially complementary to the sequence of the nucleic acid. Preferably, the antisense strand comprises a sequence which comprises at least 60%, more preferably at least 75%, most preferably at least 90% homology to a complementary sequence of the nucleic acid.

Expression of a protein can also be altered with a protein capable of binding at least a functional part of a nucleic acid encoding the protein. Binding to the functional part of a nucleic acid, at least in part, influences expression of the protein. Binding, for instance, inhibits expression of the protein. The invention, therefore, provides a the method according to the invention, wherein the compound comprises a protein capable of binding at least a functional part of a nucleic acid encoding a protein which is involved in immunocyte recruitment and/or activation of a neural pathway by an immunocyte, macrophage and/or mast cell.

According to the present invention, a compound capable of, at least in part, influencing an activity of a neural reflex pathway can be used for the preparation of a medicament for altering the motility of the gastrointestinal tract. In one aspect, the invention, therefore, provides a pharmaceutical composition for prophylactic and/or therapeutic treatment of an individual against hypomotility of the gastrointestinal tract comprising a compound capable of, at least in part, decreasing a neural reflex pathway. Preferably, the pharmaceutical composition also comprises a suitable carrier. Hypomotility of the gastrointestinal tract preferably comprises a generalized hypomotility. In a preferred embodiment, the compound comprises an antibody specifically directed against ICAM-1 and/or LFA-1, or a functional part, derivative and/or analogue thereof. In yet another preferred embodiment, the compound comprises ketotifen.

The pharmaceutical composition can be administered to an individual before he/she suffers from gastrointestinal motility disorders, such as generalized hypomotility. With prophylactic treatment, gastrointestinal motility disorder can at least partly be prevented. Gastrointestinal motility disorder cannot, for instance, occur at all or occur to a lesser extent and/or at a lesser length of time, as compared to an individual to whom the medicament is not administered.

The pharmaceutical composition can also be administered to an individual already suffering from a gastrointestinal motility disorder, like generalized postoperative ileus. In one aspect, the invention, therefore, provides a method for prophylactic and/or therapeutic treatment of an individual against hypomotility of the gastrointestinal tract, comprising administering to the individual a pharmaceutical composition according to the invention. In yet another aspect, a use of a compound capable of, at least in part, influencing an activity of a neural reflex pathway for altering the motility of the gastrointestinal tract is also provided. The compound can be used in vitro. Alternatively, the compound can be used in vivo, for instance, for treating a gastrointestinal motility disorder in an individual.

Preferably, a pharmaceutical composition or a use of the invention is provided, wherein the compound is capable of at least in part preventing immunocyte recruitment and/or activation of a neural pathway by an immunocyte, macrophage and/or mast cell. In a preferred embodiment, the compound comprises an antibody specifically directed against ICAM-1 and/or LFA-1, or a functional part, derivative and/or analogue thereof. In yet another preferred embodiment, the compound comprises ketotifen.

### BRIEF DESCRIPTION OF THE DRAWINGS

FIG. 1. Gastric emptying is delayed after abdominal surgery. Panel A shows the half emptying time (T½, open symbols) and gastric retention (Ret⁶⁴, closed symbols) as a function of time after laparotomy (L, squares) or intestinal manipulation (IM, circles). IM, performed at t=0 hour, resulted in a significant (p<0.05) increase in T½, as well as Ret⁶⁴, compared to L at t=6, 12, and 24 hours after surgery. Similar results were obtained using a caloric, solid test meal; half emptying time was significantly increased after IM, compared to mice that underwent L only (Panel B). Asterisks indicate significantly different from L using a one-way ANOVA, followed by Dunnett's multiple comparison test. Data represent mean±SEM of 8-15 mice.

FIG. 2. Ileal myeloperoxidase (MPO) activity is selectively increased at 24 and 48 hours after surgery with intestinal manipulation. MPO activity was determined in whole homogenates of ileum tissue, isolated 6, 12, 24, and 48 hours after surgery. Data are shown in Units per gram tissue, as means±SEM, n=5. MPO activity is significantly increased 24 and 48 hours after IM, compared to L or anesthesia alone (Ana), Asterisks indicate significantly different from L for each time-point using a one-way ANOVA (p<0.05), followed by Dunnett's multiple comparison test. Data represent mean±SEM of 5-8 mice.

FIG. 3. Focal leukocyte infiltrates after intestinal manipulation in the ileal muscularis tissue. Immunohistochemical staining of LFA-expressing leukocytes in transversal sections of the ileal intestinal muscularis 24 hours after IM (Panel B), but not after L alone (Panel A). MPO-activity containing leukocytes were visualized in whole mounts of ileal muscularis tissue (Panels C-F), or in homogenates of ileal muscularis tissue (Panel G) at 24 hours after surgery. Intestinal manipulation (Panel D), but not L alone (Panel C), was associated with a focal influx of MPO-containing leukocytes and an increase in MPO activity in muscularis tissue (Panel G). Anti-ICAM-1, combined with anti-LFA-1 treatment prevented leukocyte influx (Panel E). Treatment with hexamethonium did not affect the influx of MPO-staining cells (Panel F) or the amount of MPO activity in muscularis homogenates (Panel G). Asterisks indicate significant difference from L for each time-point using a one-way ANOVA (p<0.05), followed by Dunnett's multiple comparison test. Data represent mean±SEM of 5-8 mice.

FIG. 4. Gastroparesis after intestinal manipulation is prevented by blocking leukocyte infiltration or blockade of enterogastric neural pathways. Gastric emptying, determined by scintigraphic imaging of the abdomen after oral administration of semi-liquid non-caloric meal, at 6, and 24 (Panel A) hours after laparotomy only (L), or L followed by surgical manipulation of the small bowel (IM). Gastric emptying rates (k; Table in Panel B) and corresponding half-emptying times (Panel C) of semi-liquid, non-caloric, as well as caloric, solid test meals are significantly (p<0.05) increased at 6 and 24 hours after IM, compared to L. IM with a pre-operative treatment with anti-ICAM-1 and anti-LFA-1 antibodies (IM^{+MAb}) was without effect at 6 hours, but normalized gastric emptying rate k and half-emptying time at 24 hours postoperatively. Postoperative injections of hexamethonium (IM^{+hex}) or guanethidine (IM^{+gua}), normalized emptying rate k and half-emptying time at 6 hours, as well as 24 hours postoperatively. Values are averages ±SEM of 8-12 mice per treatment group. Significant differences (p<0.05), determined by one-way ANOVA with treatment group as variants, are indicated by asterisks.

FIG. 5. In vitro gastric contractility of mice that underwent intestinal manipulation is not altered. In vitro contractility of longitudinal muscle strips of gastric fundus (Panel A), and antrum (Panel B). Dosis response curves after electrical pulse stimulation (left panel), carbachol (middle panel), or prostaglandin F_{2α} (right panel) is shown. There was no difference in response in mice that underwent L only (black symbols), or IM (open symbols).

### DETAILED DESCRIPTION OF THE INVENTION

The invention will now be illustrated by the following examples which merely serve to exemplify the invention and are not intended to limit the scope of the invention.

### EXAMPLES

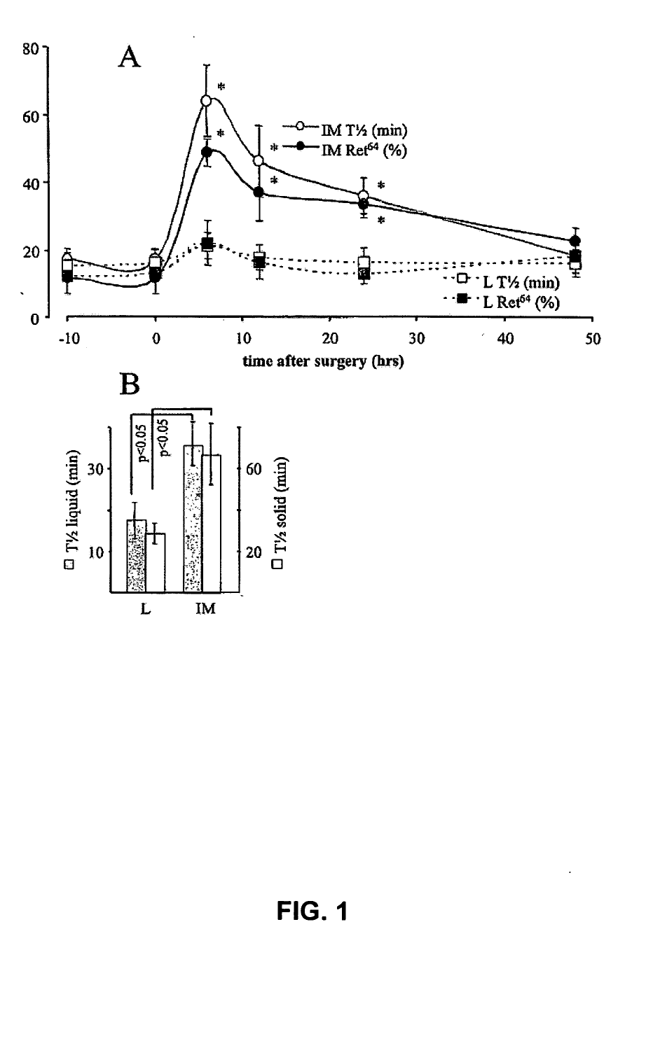

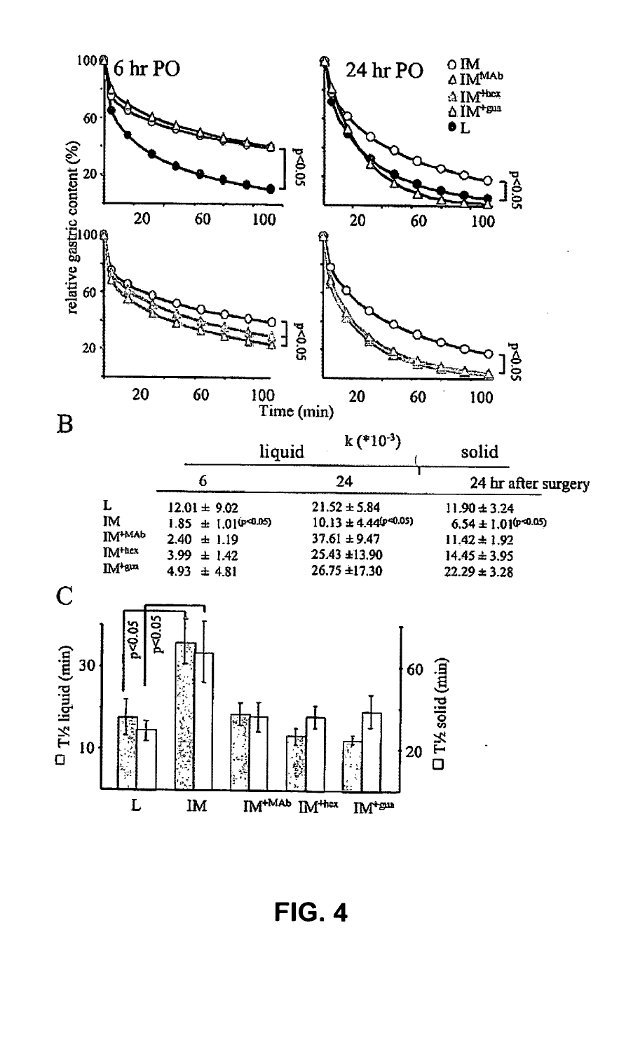

We hypothesized that inflammatory infiltrates in the myenteric plexus, recruited by bowel manipulation, are able to trigger inhibitory neuronal pathways affecting the motility of the entire gastrointestinal tract. To investigate this hypothesis, a murine model for postoperative ileus was developed. Mice underwent laparotomy (L) or laparotomy combined with intestinal manipulation (IM) over the entire length of the small intestine (12). At 6, 12, 24, and 48 hours after surgery, gastric emptying of either a non-caloric semi-liquid, or a caloric solid, test meal was measured by scintigraphic imaging (13). As shown in FIG. 1, Panel A, L alone had no effect on the rate of gastric emptying of a non-caloric liquid meal at any time-point measured after surgery. In addition, gastric emptying after L did not differ from the gastric emptying of anesthetized control mice that were not operated on (not shown). However, when laparotomy was combined with surgical manipulation of the small bowel, gastric emptying was significantly delayed (FIG. 1). The delay was especially pronounced shortly after surgery: at 6 hours postoperatively, retention of the meal 64 minutes after gavage (Ret⁶⁴) was 2.5-fold higher after IM, compared to L (p<0.05; FIG. 1), and half-emptying time (T½) was even three-fold higher (FIG. 1, Panel A). Gastric emptying after IM remained significantly delayed at 12 and 24 hours after surgery (FIG. 1, Panel A). At 48 hours postoperatively, gastric retention times and half-emptying times in L and IM-treated mice had all recovered back to normal. Similar results were obtained with a solid test meal. At 24 hours after surgery, gastric emptying of a caloric, solid test meal (13) was delayed to an extent, similar to the semi-liquid test meal: IM increased the gastric half-emptying time 2.5 fold, compared to L (FIG. 1, Panel B). In concert, emptying rate (k) of either the semi-liquid, as well as the solid, test meals was reduced to approximately half of the rate calculated after L alone (results given in FIG. 4, Panel B).

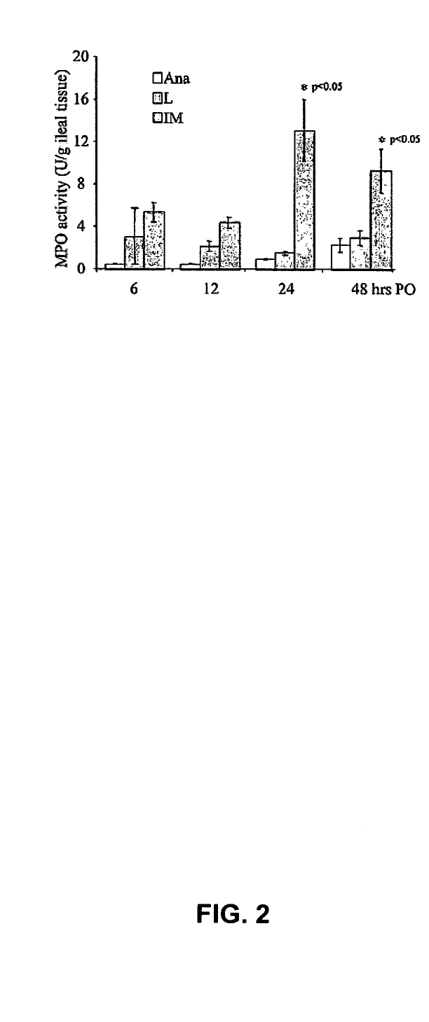

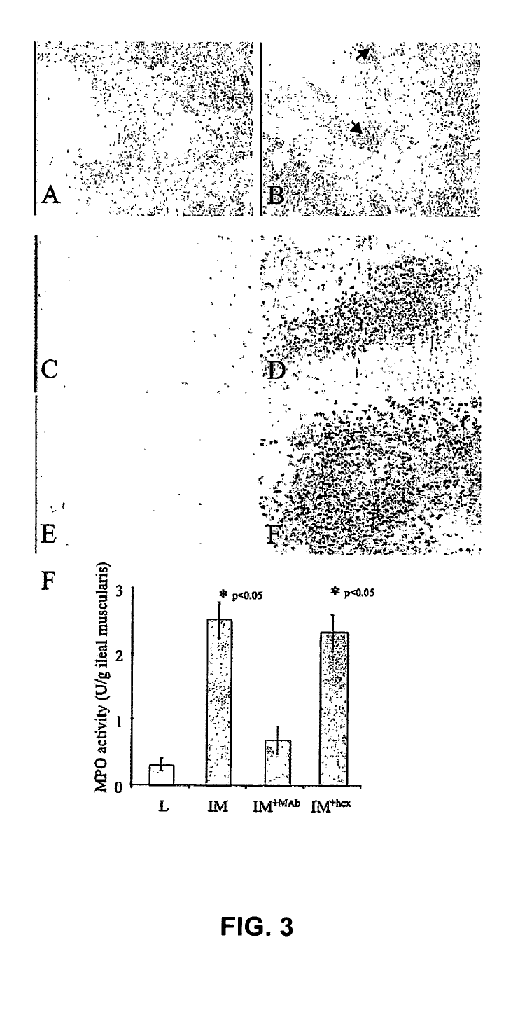

The delayed gastric emptying at 12, 24 and 48 hours after IM coincided with an enhanced activity of the neutrophil indicator myeloperoxidase (MPO) (14) in transmural ileal homogenates (15) (FIG. 2). At 24 and 48 hours after surgery, IM, but not L alone, resulted in a significant (p<0.05) increase in MPO activity measured in homogenates of transmural ileal tissue (FIG. 2), or in muscularis homogenates. No increase in MPO activity was observed at earlier time-points after surgery (FIG. 2). Histological analysis of transverse sections of ileal tissue indeed showed the presence of LFA⁺ leukocytes surrounding the myenteric plexus of the ileal muscularis 24 hours after IM, but not after L alone (16) (FIG. 3, Panels A and B). Double stainings revealed that these leukocytes were MPO⁺, but CD3⁻ and CD4⁻ (not shown). Examination of the presence of inflammatory cells containing MPO activity in whole mount preparations (FIG. 3, Panels C-F) (17) and in isolated ileal muscularis tissue (15), again confirmed the presence of leukocyte infiltrates in muscularis of manipulated ileum only (FIG. 3, Panels C and D). Importantly, no increased number of MPO-, LFA-1-, CD4-, or CD3-staining leukocytes was found in gastric fundus and antrum, nor in colonic tissue at any time-point (not shown).

In order to evaluate the role of the small intestinal infiltrate in the development of gastroparesis, IM mice received a pre-operative bolus with monoclonal blocking antibodies against either ICAM-1 alone (not shown), or in combination with LFA-1, to prevent leukocyte recruitment during the postoperative period (18). Analysis of MPO-containing leukocytes in ileal muscularis (FIG. 3, Panel C) or MPO activity in ileal muscularis homogenates (15)(FIG. 3, Panel G) at 24 hours after IM, demonstrated that antibody treatment inhibited the leukocyte recruitment down to 30% (p<0.05) of untreated ileal segments. Prevention of this inflammatory infiltrate did not ameliorate gastroparesis at 6 hours, but completely abrogated the development of gastroparesis at 24 hours after IM (FIG. 4), independent of the test meal used, revealing that the later phase of gastroparesis is mediated by an intestinal inflammatory infiltrate. The observation that the antibody regiment could not prevent gastroparesis 6 hours postoperatively is in line with the absence of an immune infiltrate at this time-point (FIG. 2).

Mast cells release a broad range of pro-inflammatory substances. In order to demonstrate that the intestinal inflammatory infiltrate upon bowel manipulation at least in part resulted from activation and/or degranulation of mast cells in the intestinal tissue or mesenterium, we pretreated mice with ketotifen (10 mg/kg p.o.), which is a mast cell stabilizer, for five days prior to surgery. Pretreatment with ketotifen decreased the postoperative recruitment of MPO-containing leukocytes with 45%, compared to treatment with vehicle (p<0.05%) at 24 hours after IM. Moreover, ketotifen treatment prevented the development of postoperative gastroparesis 24 hours after IM (not shown), indicating that mast cell degranulation during intestinal handling contributes significantly to the development of hypomotility of the gastrointestinal tract, such as postoperative ileus.

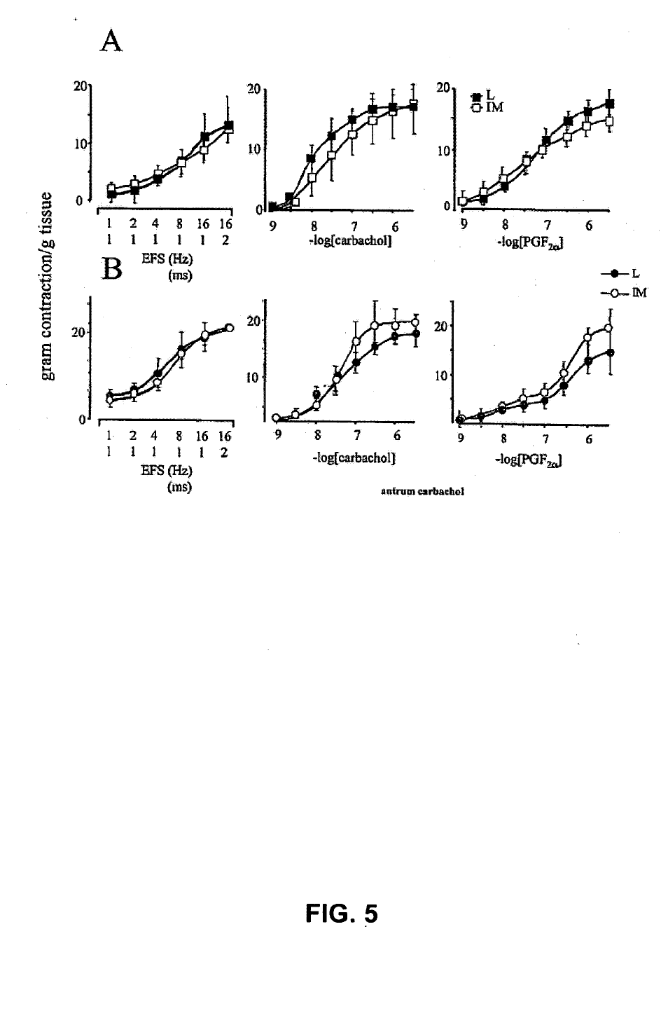

Next, we aimed to demonstrate that the gastroparesis associated with the small intestinal infiltrate was caused by activation of a neural pathway. Therefore, mice that underwent IM were treated either with a nicotinic receptor blocker, hexamethonium (hex; 1 mg/kg, 10 minutes before gastric scintigraphy), or the adrenergic blocker guanethidine (gua; 50 mg/kg, 1 hour before gastric scintigraphy) (18). Treatment with hexamethonium (FIG. 3, Panels A and B) or guanethidine (not shown) did not affect the leukocyte recruitment seen in the ileal muscularis after IM at 24 hours. However, this treatment reversed the gastroparesis partially at 6 hours, and completely at 24 hours after surgery (FIGS. 4 and 5), demonstrating that the etiology of the “acute,” as well as the “sustained” gastroparesis involves activation of an adrenergic enterogastric neural pathway.

To exclude the possibility that the delayed gastric emptying resulted from impaired local neuromuscular function, longitudinal muscle strips from the gastric fundus and antrum were mounted in organ baths (19). The isometric contractile responses to increasing concentrations of the muscarinic receptor agonist carbachol (0.1 nmol/L-3 μmol/L), and of prostaglandin F_{2α} (0.1 nmol/L-3 μmol/L) was determined. Intestinal manipulation did not affect the dose-dependent contractile response upon stimulation of gastric muscle strips with prostaglandin F_{2α} or carbachol, compared to mice that underwent laparatomy alone (FIG. 5). In addition, nerve stimulation-evoked (0.5-16 Hz, 1 ms pulse duration, 10 s pulse trains) contractions in fundus and antrum from IM and L mice were not significantly different (FIG. 5). Together, these results demonstrate that the delayed gastric emptying does not result from an impaired local gastric neuromuscular function, but rather results from inhibitory extrinsic neural input.

We have, amongst other things, established a causal relationship between bowel manipulation, leukocyte infiltration into the intestinal muscularis, and delayed gastric emptying. Intestinal manipulation, but not laparatomy or anesthesia alone, delayed gastric emptying up to 24 hours after surgery, an effect mediated by inhibitory extrinsic neuronal input. In contrast to the first 6 hours, the development of gastroparesis at 24 hours was dependent on the influx of intestinal leukocytes in the manipulated small intestine, and could be prevented by pretreatment with ICAM-1 antibodies, LFA-1 antibodies, and/or ketotifen. Importantly, no infiltrates were found in the stomach and colon that were not manipulated.

### REFERENCES AND NOTES

1. Boeckxstaens, G. E., D. P. Hirsch, A. Kodde, T. M. Moojen, A. Blackshaw, G. N. Tytgat, and P. J. Blommaart. 1999. Activation of an adrenergic and vagally-mediated NANC pathway in surgery-induced fundic relaxation in the rat. Neurogastroenterol. Motil. 11:467.
2. Boeckxstaens, G. E., M. Hollmann, S. H. Heisterkamp, P. Robberecht, W. J. de Jonge, R. M. van Den Wijngaard, G. N. Tytgat, and P. J. Blommaart. 2000. Evidence for VIP(1)/PACAP receptors in the afferent pathway mediating surgery-induced fundic relaxation in the rat. Br. J Pharmacol. 131:705.
3. De Winter, B. Y., G. E. Boeckxstaens, J. G. De Man, T. G. Moreels, A. G. Herman, and P. A. Pelckmans. 1997. Effect of adrenergic and nitrergic blockade on experimental ileus in rats. Br. J. Pharmacol. 120:464.
4. Kalff, J. C., B. M. Buchholz, M. K. Eskandari, C. Hierholzer, W. H. Schraut, R. L. Simmons, and A. J. Bauer. 1999. Biphasic response to gut manipulation and temporal correlation of cellular infiltrates and muscle dysfunction in rat. Surgery. 126:498.
5. Barquist, E., B. Bonaz, V. Martinez, J. Rivier, M. J. Zinner, and Y. Tache. 1996.

Neuronal pathways involved in abdominal surgery-induced gastric ileus in rats. Am. J. Physiol. 270:R888.
6. Prasad, M., and J. B. Matthews. 1999. Deflating postoperative ileus. Gastroenterology. 117:489.
7. Raybould, H. E., R. J. Gayton, and G. J. Dockray. 1988. Mechanisms of action of peripherally administered cholecystokinin octapeptide on brain stem neurons in the rat. J. Neurosci. 8:3018.
8. Kalff, J. C., W. H. Schraut, R. L. Simmons, and A. J. Bauer. 1998. Surgical manipulation of the gut elicits an intestinal muscularis inflammatory response resulting in postsurgical ileus. Ann. Surg. 228:652.
9. Kalff, J. C., T. M. Carlos, W. H. Schraut, T. R. Billiar, R. L. Simmons, and A. J. Bauer. 1999. Surgically induced leukocytic infiltrates within the rat intestinal muscularis mediate postoperative ileus. Gastroenterology. 117:378.
10. Holte, K., and H. Kehlet. 2000. Postoperative ileus: a preventable event. Br. J Surg. 87:1480.
11. De Winter, B. Y., G. E. Boeckxstaens, J. G. De Man, T. G. Moreels, A. G. Herman, and P. A. Pelckmans. 1998. Differential effect of indomethacin and ketorolac on postoperative ileus in rats. Eur. J Pharmacol. 344:71.
12. IM. Mice (female BalB/C, Charles River) were kept under environmentally controlled conditions (light on from 8:00 a.m. to 8:00 p.m.; water and rodent none-purified diet ad lib; 20-22° C., 55% humidity). Mice were used at 6-10 weeks of age. The surgical procedures were carried out as follows: mice were fasted overnight before surgery, and were anesthetized by an i.p. injection of a mixture of ketamine (100 mg/kg) and xylazine (20 mg/kg). Mice were divided in three groups of 10-12 animals each: mice undergoing 1-anesthesia (Ana), 2-anesthesia and laparotomy (L), 3-laparotomy and intestinal manipulation (IM). A midline abdominal incision was made, and the peritoneum was opened over the linea alba. The small bowel was carefully exteriorized and manipulated by “running” through its entire length for 5 minutes using sterile moist cotton applicators. After the surgical procedure, mice were closed by a continuous two-layer suture (Mersilene, 6-0 silk). After closure, mice were allowed to recover for 4 hours in a heated (32° C.) recovery cage. After 4 hours, mice were completely recovered from anesthesia. At 6, 12, 24, and 48 hours after surgery, gastric emptying was measured.
13. Emptying. We first established that the anesthetics used did not alter gastric emptying in control mice, either xylazine (20 mg/kg) used alone, or in combination ketamine (100 mg/kg). Furthermore, the handling of mice for scanning necessary to determine gastric emptying was restricted to once every 16 minutes during a measurement period of 80 minutes to reduce handling stress. Mice were fed 0.1 mL of a semi-liquid meal by intragastric gavage, consisting of 1 mL 30 mg/ml methylcellulose dissolved in water, and 1 mL of a solution containing 200 MBq 99 mTc/mL. Caloric, solid test meals were prepared by baking 4 g of egg-yolk containing 400 MBq of 99 mTc. Mice were offered 100 mg of the baked egg-yolk, which was completely consumed within 1 minute. Immediately after administration of the meal, mice were held manually under a large field of a view gamma camera fitted with a medium energy collimator and interfaced to a nuclear medicine computer system (Hermes). Twenty percent energy windows were set with peaks set at 141 KeV for 99 mTc. Static images of the entire abdominal region were obtained for 30 seconds at 16 minute intervals for 96 minutes (semi-liquid) or 112 minutes (solid). The gastric emptying curves were analyzed using a modified power exponential function y(t)=1−(1−ekt)b, where y(t) is the fractional meal retention at time t, k is the gastric emptying rate in minute-1, and b is the extrapolated y-intercept from the terminal portion of the curve.
14. Witko-Sarsat, V., P. Rieu, B. Descamps-Latscha, P. Lesavre, and L. Halbwachs-Mecarelli. 2000. Neutrophils: molecules, functions and pathophysiological aspects. Lab. Invest. 80:617.
15. MPO. Tissue myeloperoxidase (MPO) activity was determined as follows: either full thickness ileal segments, or isolated ileal muscularis, was blotted dry, weighed, and homogenized in a 20 times volume of a 20 mmol/L potassium phosphate buffer, pH 7.4. The suspension was centrifuged (8000*g for 20 minutes at 4° C.) and the pellet was taken up in 1 mL of a 50 mmol/L potassium phosphate buffer, pH 6.0, containing 0.5% of hexadecyltrimethylammonium (HETAB) bromide and 10 mmol/L EDTA, and stored in 0.1 mL aliquots at −70° C. until analysis. 50 μL of the appropriate dilutions was added to 445 μL of assay mixture, containing 0.2 mg/mL tetramethylbenzidine in 50 mg potassium phosphate buffer, pH 6.0, 0.5% HETAB, and 10 mmol/L EDTA. The reaction was started by adding 5 μL of a 30 mmol/L H2O2 to the assay mixture, and the mixture was incubated for 3 minutes at 37° C. After 3 minutes, 30 μL of a 300 μg/mL catalase solution was added to each tube, and tubes were placed on ice for 3 minutes. The reaction was ended by adding 2 mL of 0.2 mol/L glacial acetic acid and incubating at 37° C. for 3 minutes. Absorbance was read at 655 nm. One unit of MPO activity was defined as the quantity required to convert 1 μmol of H2O2 to H2O per minute at 25° C., and activity was given in Units per gram tissue.
16. IHC. Immunohistochemistry was performed as follows: after rehydration, endogenous peroxidase activity in the sections was eliminated by incubation for 30 minutes in PBS (10 mM sodium phosphate, 150 mM sodium chloride, pH=7.4) and 50% methanol, containing 3% (wt/vol) hydrogen peroxide. Non-specific protein-binding sites were blocked by incubation for 30 minutes in TENG-T buffer (10 mM Tris, 5 mM EDTA, 150 mM sodium chloride, 0.25% gelatin, 0.05% Tween-20, pH=8.0). Serial sections were incubated overnight with an appropriate dilution of rat monoclonal antibodies against LFA, CD3, and CD4. The indirect unconjugated peroxidase-anti-peroxidase technique [Sternberger, 1970 #514] was used to visualize binding of the primary antibodies, with AEC as a substrate, dissolved in Sodium Acetate buffer (pH=7.4) to which 0.01% hydrogen peroxide was added.
17. WM. Whole mounts of ileal segments were prepared as previously described with slight modifications [Kalff, 1998 #9]. In short, mid-ileal segments were quickly excised and mesentery was carefully removed. Intestinal segments were cut open along the mesentery border, fecal content was washed out in ice-cold PBS, and segments were pinned flat in a glass dish filled with pre-oxygenated Krebs-Ringer solution. Mucosa was removed and the remaining full-thickness sheet of muscularis externa was fixed for 10 minutes in 100% ethanol. Muscularis preparations were kept on 70% ethanol at 4° C. until analysis.
18. Treatments. A pretreatment bolus of monoclonal antibodies 1A29 (anti-ICAM-1; 4.5 mg/kg) and WT.1 (anti-LFA-1; 2.25 mg/kg), dissolved in dialyzed saline (0.9% sodium chloride), was given by intraperitoneal injection 1 hour before surgery. The antibody doses used has been shown to block ICAM-1 receptors for at least 24 hours (ref). Hexamethonium (1 mg/kg i.p. in sterile 0.9% sodium chloride) or guanethidine (50 mg/kg, i.p.) was administered 10 minutes, or 1 hour resp. before the gastric emptying tests at 6 hours or 24 hours postoperatively. The surgical procedures and treatments resulted in no deaths or major surgical complications such as hemorrhage, peritonitis, or perforation.
19. Contractility. In vitro contractility measurements were performed as follows: After removal of the mucosa, two longitudinal muscle strips (10×5 mm) of the gastric fundus and antrum were mounted in organ baths (25 ml) filled with Krebs-Ringer solution maintained at 37° C. and aerated with a mixture of 5% CO2 and 95% O2. At the end of the experiment, muscle strips were blotted and weighed. One end of the muscle strip was anchored to a glass rod and placed between two platinum electrodes. The other end was connected to a strain gauge transducer (Statham, UC2) for continuous recording of isometric tension. The muscle strips were brought to their optimal point of length-tension relationship using 3 μmol/L acetylcholine and then allowed to equilibrate for at least 60 minutes before experimentation. Experimental protocols. Neurally mediated contractions of the muscle strips of both the gastric fundus and antrum were induced by means of electrical field stimulation (EFS; 0.5-16 Hz, 1 and 2-ms pulse duration, 10-s pulse trains). Responses were always measured at the top of the contractile peak. In a second series of experiments, contractions were evoked by the muscarinic receptor agonist carbachol (0.1 nmol/L to 3 μmol/L) and prostaglandinF2a (0.1 nmol/L-3 μmol/L). Between the responses to the different contractile receptor agonists, tissues were washed four times with an interval of 15 minutes. Contractions were expressed in grams of contraction per mg of tissue weight.

1. A method of altering the motility of a subject's gastrointestinal tract, said method comprising:
administering to the subject a medicament comprising a compound capable of at least in part influencing an activity of a neural reflex pathway.

2. The method according to claim 1, wherein the activity is decreased.

3. The method according to claim 1 for prophylaxis and/or treatment of hypomotility of the gastrointestinal tract.

4. The method according to claim 3, wherein the hypomotility comprises a generalized hypomotility.

5. The method according to claim 1, wherein the activity of the neural reflex pathway is decreased by at least in part preventing stimulation of the neural reflex pathway by an immunocyte.

6. The method according to claim 1, wherein the activity of the neural reflex pathway is decreased by at least in part preventing immunocyte recruitment.

7. The method according to claim 5, wherein the immunocyte comprises a leukocyte.

8. The method according to claim 1, wherein the activity of said neural reflex pathway is decreased by, at least in part, preventing stimulation of said pathway by a macrophage and/or mast cell.

9. The method according to claim 1, wherein the activity of the neural reflex pathway is decreased by, at least in part, decreasing the release of a pro-inflammatory mediator by a macrophage and/or mast cell.

10. The method according to claim 1, wherein said compound is capable of specifically binding at least a functional part of a protein involved in immunocyte recruitment and/or activation of a neural pathway by an immunocyte, macrophage and/or mast cell.

11. The method according to claim 1, wherein said compound comprises a nucleic acid that binds at least a functional part of a nucleic acid encoding a protein involved in immunocyte recruitment and/or activation of a neural pathway by an immunocyte, macrophage and/or mast cell.

12. The method according to claim 1 wherein said compound comprises a protein capable of binding at least a functional part of a nucleic acid encoding a protein which is involved in immunocyte recruitment and/or activation of a neural pathway by an immunocyte, macrophage and/or mast cell.

13. The method according to claim 10, wherein the protein is selected from the group of ICAM-1, LFA-1, statin, PSGL-1, L-selectin, L-selectin receptor, P-selectin, P-selectin receptor, α4-β7 integrin, MadCAM, CD44, substance P, NK-1 receptor, CGRP, CGRP-receptor, VIP, VIP-receptor, neutral endopeptidase, neurotensin, neurotensin receptor, nerve growth factor, neurotropin-3, and combinations of any thereof.

14. The method according to claim 1, wherein said compound comprises an antibody specifically directed against ICAM-1 or a part or analogue thereof.

15. The method according to claim 1, wherein said compound comprises an antibody specifically directed against LFA-1, or a part or analogue thereof.

16. The method according to claim 8, wherein the activity of the neural reflex pathway is decreased by at least in part preventing release of histamine, tryptase, and/or tachykinuns by a macrophage and/or mast cell.

17. The method according to claim 9, wherein said pro-inflammatory mediator is selected from the group consisting of histamine, trypsine, tryptase, chymase, 5-hydroxytryptamine, IL8, TNFα, and a combination of any thereof.

18. The method according to claim 1, wherein said compound comprises ketotifen.

19. A pharmaceutical composition for prophylactic and/or therapeutic treatment of an individual against hypomotility of the gastrointestinal tract comprising a compound capable of, at least in part, decreasing a neural reflex pathway.

20. A method for prophylactic and/or therapeutic treatment of an individual against hypomotility of the gastrointestinal tract, comprising administering to said individual a pharmaceutical composition according to claim 19.

21. A method of altering the motility of a subject's gastrointestinal tract, the method comprising: administering to the subject a compound capable of at least in part influencing an activation of a neural reflex pathway for altering the motility of the gastrointestinal tract.

22. A method of altering the motility of a subject's gastrointestinal tract, the method comprising: administering to the subject a compound capable of specifically binding at least a functional part of a protein involved in immunocyte recruitment.

23. A method of altering the motility of a subject's gastrointestinal tract, the method comprising: administering to the subject a compound capable of specifically binding at least a part of a protein involved in activation of a neural pathway by an immunocyte, macrophage and/or mast cell.

24. The pharmaceutical composition of claim 19, wherein said compound is capable of at least in part preventing immunocyte recruitment and/or activation of a neural pathway by an immunocyte, macrophage and/or mast cell.
